(19) United States
(12) Patent Application Publication
(10) Pub. No.: US 2011/0208084 A1
(43) Pub. Date: Aug. 25, 2011

(54) METHOD AND APPARATUS FOR BRAIN DAMAGE DETECTION

(76) Inventors: Fernando Seoane Martinez, Goteborg (SE); Kaj Lindecrantz, Vastra Frolunda (SE)

(21) Appl. No.: 13/061,960

(22) PCT Filed: Sep. 9, 2009
(86) PCT No.: PCT/SE2009/051004

§ 371 Date: Mar. 28, 2011

(30) Foreign Application Priority Data
Sep. 9, 2008 (SE) 0801937-4

(51) Int. Cl. A61B 5/053

(52) U.S. Cl. 600/547

(57) ABSTRACT

The present invention comprises method and apparatus for detecting injury resulting in pathological processes affecting tissue within a portion of the body in a mammal, particularly a human brain. Said method comprises the steps of applying a first and a second pair of electrodes around the periphery of the portion, generating an alternating current at a known current level and applying said current between the first pair of electrodes, detecting and measuring the alternating voltage developed between the second pair of electrodes, and calculating the impedance of said portion. Further, the alternating current is applied between the first pair of electrodes in a series of increasing frequencies ranging within a known spectrum, and the resistance and the reactance for each frequency are detected and plotted against said frequency. The electrical impedance of said portion is calculated for each frequency and plotted into an impedance plot. Said resistance-reactance-and impedance-plots are finally analyzed, and any notable changes compared to normal spectrum profiles and plots are detected and evaluated.

### TECHNICAL FIELD

The present invention relates to a method for detecting status of brain tissue resulting in pathological processes affecting tissue within a portion of the body in a mammal, including a human, comprising the steps of applying a first and a second pair of electrodes around the periphery of the portion, generating an alternating current at a known current level, applying said current between the first pair of electrodes, detecting and measuring the alternating voltage developed between the second pair of electrodes, and calculating the impedance of said portion.

### BACKGROUND ART

It is previously known to measure the electrical impedance of the body for medical diagnosis and in various clinical practices. For instance, the impedance has been used to assess total body fat content, detect lymphoedema and to monitor cardiac activity. The dielectrical properties of biological tissue depend on the tissue intrinsic composition and the tissue structure. If any of these elements change, the impedance of the tissue changes as well and since the electrical impedance of a material is directly proportional to the dielectric properties, the changes propagates to the electrical impedance. Thus, any changes in tissue structure, e.g. due to oedema or inner bleeding, are possible to be detected through the spectroscopy study of the dielectrical properties of said body part.

In patent U.S. Pat. No. 5,807,270 an impedance monitoring equipment and a method for use thereof are disclosed. The patent primarily relates to monitoring of oedema in tissues, and particularly intracellular oedema within the brains of mammals including humans. Described in a broad aspect the invention comprises a monitor which has a current source supplying a one microampere AC square waveform at a low frequency of 200 Hz through an electrode pair placed at a periphery around a body portion meant to be monitored. At the same periphery are located a pair of sensing electrodes repeatedly sensing and calculating the electrical impedance through said portion. The electrical impedance is displayed graphically over time, and the readings are to be interpreted in the light of past trends, that is, the results are expressed as percentage change as referred to an initial value.

When using frequencies for monitoring as in said patent, the imaginary part of the electrical impedance, the reactance, may or may not change, and in the case any change occurs it is too small to be detected. Thus, the impedance will consist solely of its real part, the resistance meaning the above described patent focuses on the monitoring and recognition of changes in electrical impedance at frequencies where the impedance virtually consists of the resistance. However, to evaluate the status of tissue hemorrhagic, ischemic, etc., the actual causes that lead to a change in resistance are impossible to detect. Said method may be useful for indication of tissue change, but a change in resistance does not reveal much about the tissue per se.

### DISCLOSURE OF THE INVENTION

It is an object of the present invention to provide a method and apparatus for solving the above mentioned problems and improve the methods for measurement of bioimpedance for brain monitoring, for assessment of brain tissue status and for detection and evaluation of changes and/or abnormalities in brain tissue composition. This is accomplished through a method for detecting status of tissue resulting in pathological processes affecting tissue within a portion of the body in a mammal, including a human. Said method comprises the steps of applying a first and a second pair of electrodes around the periphery of the scalp surrounding the brain portion, generating an electrical stimulus, e.g. in the form of a current or a voltage, and applying said current between the first pair of electrodes thus creating a current through said brain portion, detecting and measuring the alternating voltage developed between the second pair of electrodes, and calculating the impedance of said brain portion. Furthermore, the electrical stimulus is applied between said first pair of electrodes at different frequencies ranging within a known spectrum, and the resistance and the reactance for each frequency within said spectrum are detected and preferably, but not necessarily, plotted. The electrical impedance of said portion is calculated for each frequency and analyzed, and any notable changes or abnormalities compared to normal spectrum profiles are detected and evaluated.

According to one aspect of the invention the electrical impedance of said portion is calculated for each frequency and plotted into resistance- and reactance spectrums as well as into an impedance plot. Comparison may thus be performed in a graphical manner by looking at profiles of different spectrums and analyze with respect to possible shifts in spectral moments and abnormalities indicating a lesion. However, other ways of analyzing spectrums are possible, for instance a numerical presentation of collected data, or comparison of certain key features of a spectrum.

Measurements in a broad frequency band where the impedance is completely complex means that both resistance and reactance will have noticeable magnitude which will enable a better detecting of possible divergence from normal values. This is even more important considering that different types of injures (e.g. bleeding or swelling) will lead to different types of changes in tissue structure and composition, and depending on what type of change that occurs the resistance and the reactance will be affected in different ways.

During bleeding, blood from the cardiovascular system leaks through broken vessels to the extracellular space. This increases the amount of extracellular fluid and since blood is a part of the extracellular fluid the conductivity of the extracellular fluid is modified as well. The combination of these changes (and/or abnormalities) in morphology and composition of the extracellular fluid cause the resistance and the reactance to increase at most frequencies, and the present method and apparatus will provide a way to detect said changes and/or abnormalities in brain tissue composition.

In the case of cell swelling, the size of the cells constituting the tissue increases by intake of water from the extracellular space. The increasing of the intracellular space and the surface of the cell membrane in each cell, together with the decreasing of the extracellular space and fluid causes several changes in the dielectric properties of the tissue. The electrical conductivity of the intracellular fluid and the conductance of the intracellular space increases, the conductivity of the extracellular fluid and the conductance of the extracellular space decreases and the capacitance caused by the cell membrane of the cells increases as well. The cell membrane is able to accumulate electrical charges acting as a capacitor. Said changes modifies both the resistance and the reactance, and thus also the impedance spectrum.

At cell swelling, the resistance increases remarkably at low frequencies and decreases slightly at high frequencies. The reactance increases at all frequencies in a way that causes a noticeable shift of the characteristic frequency towards lower values.

It is understood that the characteristic frequency is the frequency at which the reactance obtains its maximum value; during cell swelling the electrical reactive character of the tissue increases since the cellular membranes act as a capacitor.

The changes in resistance- and/or reactance spectrums of a body portion, preferably a portion of a brain, could rapidly and conveniently reveal the type of damage to be expected. Analyzing said resistance and reactance acquired at different frequencies thus allows for a primary detection as to whether the examined body portion is normal or suffers from abnormal changes, e.g. ischemic injuries. Monitoring and/or analyzing key features of a spectrum, such as the characteristic frequency, may provide a quick way of detecting abnormal changes indicating a lesion within the brain portion. If for instance a notable shift of the characteristic frequency occurs this may be instantly discovered and attended to.

Being preferably non-invasive and preferably being carried out with portable and mobile equipment, the method has the advantage that it can be used both in- and outside of a controlled environment, e.g. in an ambulance on the way to a hospital, in the emergency room or in any other place outside of a hospital.

Electrical impedance can be measured by applying an electrical stimulus to object under study and measuring the electrical response caused by the applied stimulus. The electrical stimulus can be applied in several electromagnetic manners through a stimulating channel: applying voltage, injecting current, etc. Therefore the electrical stimulus can be caused by a voltage source or current source. Independently of the form of the electrical stimulus, voltage or current, the stimulus will cause an electrical current to flow through the object under study.

The caused electrical current will flow through the object under study electrical as a response that can be measured through voltage or current measurements. The response to an electrical stimulus can be measured by a single measurement channel or by several measurement channels. The latter is known as multichannel measurement.

The transfer impedance between the stimulating channel and the measurement channel can be estimated from the stimulating signal and the measured response signal by several mathematical methods: Fourier analysis, Sine Correlation, Total Least Squares, etc.

An electrical impedance spectroscopy measurement contains measurements at more than one frequency defining a frequency range. The impedance in the frequency range can be measured by applying the electrical stimulus at one frequency at a time, sweeping through the frequency range. In addition the electrical stimulus might be constituted by more than one frequency, this way it is possible obtain the impedance information at more than one frequency at a time. Examples of the later are stimulus with a step function, square, multisine or white noise waveforms.

According to another aspect, the invention comprises a method for detecting brain injury where the amplitude of the current generated by the electrical stimulus is preferably not larger than 1 milliAmpere RMS and complies with applicable normative for safety limits. For instance nowadays for humans the IEC60601.

The applied frequency of said electrical stimulus may be covering a frequency band between a value of 1 kHz, more preferred, of 5 kHz, but even more preferred of 20 kHz, and a value of 2 MHz, preferably 1 MHz. The choice of said frequency band will contain a significant part of the frequency window known as β-dispersion ensuring that the contribution of the extracellular space, cell membranes and intracellular space to the tissue impedance can be measured. Preferably, the frequency band will also cover said characteristic frequency, being both below and above the characteristic frequency. Moreover, when a current is injected at these high frequencies the body will not respond to the charge accumulation. In addition, the discharge occurs quicker than the cells can detect and react to it, leading to that no stimulus is ever created and no harmful cellular response will be triggered.

The use of high frequencies also brings the advantage that no interference will occur with other electrical measurement devices, such as EEG or the like.

According to another aspect the invention comprises an apparatus for carrying out the method as previously described, capable of determining the electrical impedance and recognizing changes in electrical impedance of a tissue, e.g. a damaged body portion, preferably a brain portion. Said apparatus is preferably mobile and is preferably comprises a portable control unit. Depending on the place of detection (in an ambulance, at a patient's home, within the clinic etc.) said apparatus can be brought closer to the person in need of damage detection, e.g. as a part of the equipment within an ambulance.

The apparatus further comprises means capable of generating an electrical stimulus. It is to be understood that said electrical stimulus could equal an alternating current at known frequency and amplitude, or a voltage that when applied over e.g. a portion of a brain results in an electrical current through said portion.

Moreover, the apparatus comprises means capable of measuring the voltage drop produced across the second pair of electrode means as a consequence of the passage of the alternating current through the body portion.

Furthermore, the apparatus comprises means for estimating the electrical impedance of said body portion and means capable of detecting and measuring the change in electrical impedance in said body portion as a result of changed frequency of the applied alternating current. Preferably said means for calculating the electrical impedance is comprised by a central processing unit and an analyzing software. Preferably, the apparatus also comprises means for graphical display of calculated electrical impedance. Preferably the apparatus includes the capability to perform multichannel measurements of electrical impedance.

### BRIEF DESCRIPTION OF THE DRAWINGS

The method and apparatus will hereinafter be described in more detail with reference to the accompanying drawings and diagrams. The following descriptions should be considered as preferred forms only, and are not decisive in a limiting sense.

FIG. 1: illustrates a portable control unit comprising a first and a second pair of electrodes applied around a periphery of a brain portion,

FIG. 2: illustrates a block diagram of preferred damage assessment process,

FIG. 3: is a graph showing resistance spectrum of normal vs. bleeding tissue,

FIG. 4: is a further graph showing reactance spectrum of normal vs. bleeding tissue,

FIG. 5: is a further graph as in FIG. 3 and FIG. 4 showing the impedance plot of normal vs. bleeding tissue,

FIG. 6: is a graph showing normalized resistance spectrum of normal tissue vs. tissue undergoing cell swelling,

FIG. 7: is a further graph showing normalized reactance spectrum of normal tissue vs. tissue undergoing cell swelling,

FIG. 8: is a further graph as in FIG. 6 and FIG. 7 showing the normalized impedance plot of normal tissue vs. tissue undergoing cell swelling,

FIG. 9a-c: represent schematic views showing an example of electrode array mesh and how the impedance measurement channels are established between the injecting and sensing electrodes of the electrode array,

FIG. 10: illustrates an electrode array mesh and a possible distribution of the electrodes for measuring brain impedance from the surface of the head, and

FIG. 11: illustrates a textile cap as shown in FIG. 10 applied around a periphery of a head surface.

### DETAILED DESCRIPTION OF THE INVENTION

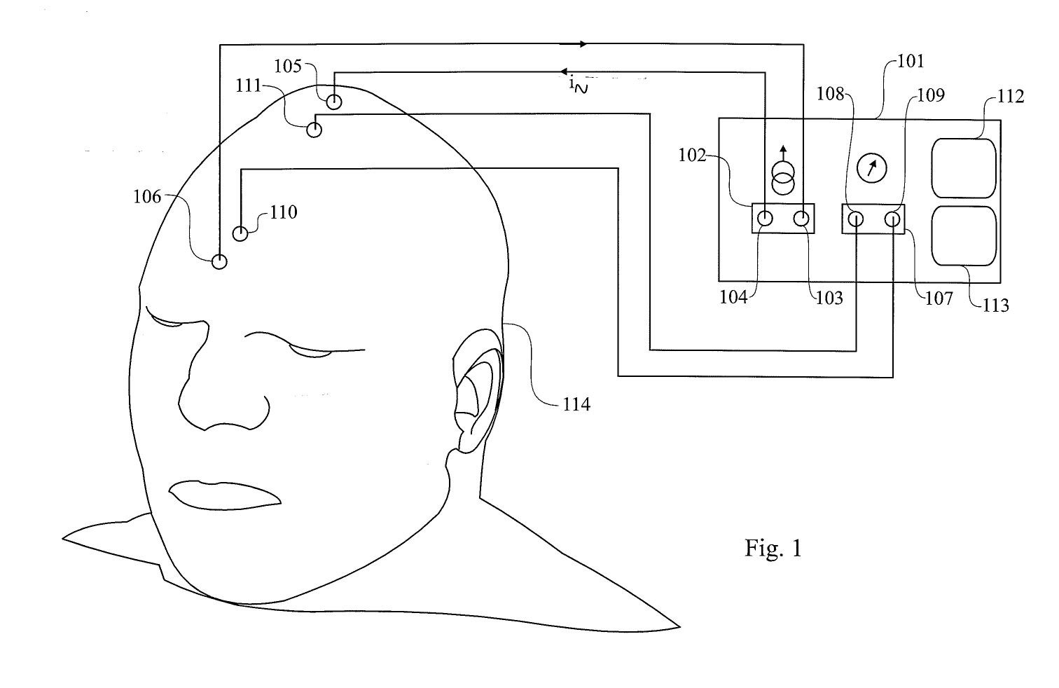

FIG. 1 illustrates a portable control unit 101 comprising means for generating an alternating current 102 with at least one pair of outlets 103, 104 coupled to at least one of pair of current injecting electrodes 105, 106.

Said control unit 101 further comprises means for voltage measuring 107 and at least one pair of inlets 108, 109 coupled to at least one of each pair of potential-sensing electrodes 110, 111.

A pair of current injecting electrodes together with a pair of potential sensing electrodes represents one measurement channel for measuring electrical bioimmitance: e.i. bioimpedance (EBI) and admittance. It is understood that the present invention may comprise one measurement channel, as in FIG. 1, or more than one measurement channel.

In a preferred aspect a spectral measurement for brain damage detection is taken by that an operator puts said current injecting electrodes 105, 106 in direct contact with the scalp or head skin and around a periphery of the body portion to be examined 114, preferably a brain portion.

It is understood that said “operator” could equal a doctor, a paramedic or any other medical practitioner in charge of the detection process.

Preferably, an electrical stimulus in the form of an alternating current is injected into the brain portion through the first pair of electrodes 105, 106 at different frequencies applied within the range of preferably 5 kHz-2 MHz. It is understood that said electrical stimulus could be applied in the form of a voltage over the portion to be measured.

When applied in humans, the amplitude of the generated current is preferably not larger than 1 milliAmpere RMS for frequencies over 10 kHz. For frequencies within the frequency range between 1 and 10 kHz the maximum amplitude will never be larger than 0.1 times the frequency and preferably not below 100 microAmperes RMS. For instance 500 microAmperes at 5 kHz, 800 microAmperes at 8 kHz and 100 microAmperes at 1 kHz. If medical regulations changes setting the limits for electrical stimulation lower or higher than the current values indicated here the amplitude of the injected current can be adjusted to comply the medical regulations.

The voltage response is measured in a continuous way by said second pair of potential-sensing electrodes 110, 111. Said potential-sensing electrodes are put in direct contact with the scalp around a periphery of the brain portion to be examined 114. Said injection of alternating current is done automatically either by sweeping, multisine or an equivalent method. Sweeping refers to a successive increase or decrease of the frequency of the alternating current in between said frequency range, and multisine refers to an applied stimulus containing several frequencies and injecting them all at once. The increase of frequency could be done either manually by the operator, or automatically by a storage injection protocol pre-programmed on the apparatus.

From the stimulating signal and the response measurements the impedance is estimated preferably using the Fourier Transform of the acquired signals. Typically the stimulating signal will be electrical current and the response will be voltage measurement.

Once a series of frequencies has been injected through said brain portion 114 through the current injection electrodes 105, 106, and the corresponding voltage has been measured by the potential-sensing electrodes 110, 111 the spectral impedance data (equalling values for resistance and for reactance) is processed by a central processing unit (not shown) and a software preferably incorporated into the portable control unit 101 within the same housing as said means for generating alternating current 102 and as said means for voltage measuring 107. Preferably the portable control unit 101 further includes means 113 for calculating the impedance of the measured brain portion 114, and means for generating the results on a display 112 thus allowing for reading and interpretation of the results, preferably in the form of an impedance plot as shown in the accompanying graphs and as described in the following figures. Depending on the clinical scenario said display 112 could be a regular PC-monitor, an external monitor or a display embedded in the portable control unit 101. The operator will have the whole spectral measurement and spectroscopy analysis results to assess on the brain tissue status.

It is understood that different subjects (e.g. adult, infant or any mammal) will have a different absolute values of calculated impedance, and thus also a different spectrum profile. The obtained impedance spectrum will further depend on the position of the electrodes when carrying out the measurements.

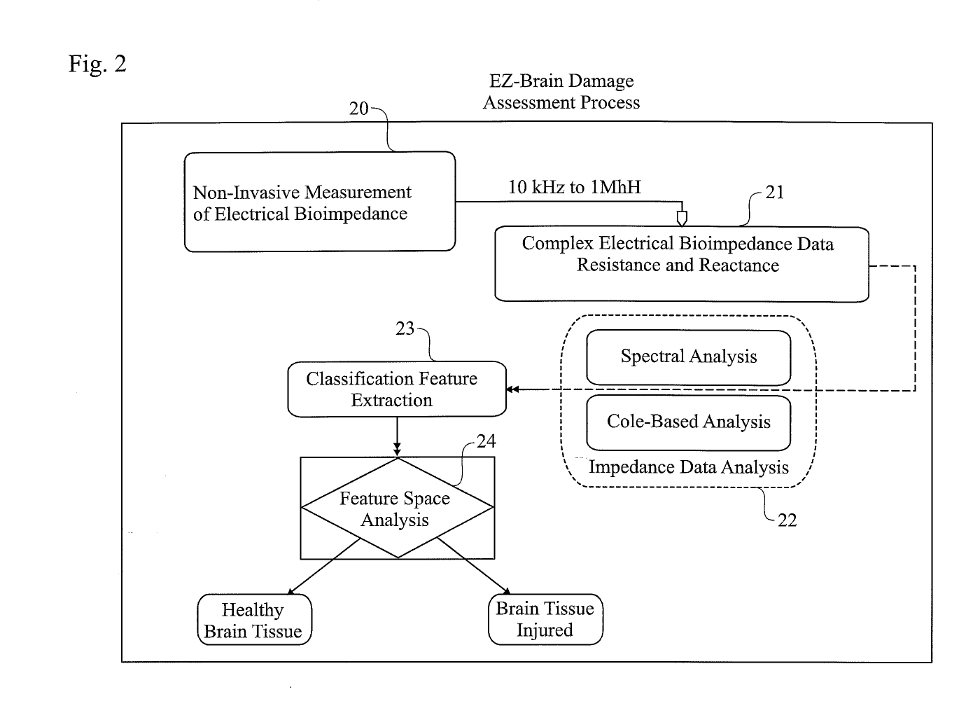

FIG. 2 is a block diagram showing the assessment process of brain damage detection starting with performance of non-invasive measurement of the electrical impedance 20 of a body portion 114 as described in FIG. 1. Several measurements are carried out using frequencies ranging between 5 kHz and 2 MHz obtaining the spectrum of the complex impedance data 21, meaning: resistance and reactance for each applied frequency. Further the processor unit will perform signal analysis of the impedance spectrum 22. The signal analysis performs a spectrum analysis of the resistance, reactance, impedance module and phase. A model-based analysis can be performed through Cole parameters as well as lumped circuit values or Cole plot analysis, hereby introduced by the way of references (K. S. Cole 1940). In addition Impedance index analysis (S. Ollmar 1998) can be also performed. The signal analysis will produce several results in form of parameters and indexes that will be used as candidate features for identification and classification 23. A final feature space analysis 24 will provided an indication of the status of examined body portion 114, allowing the clinical expert for assessment of a potential damage therein.

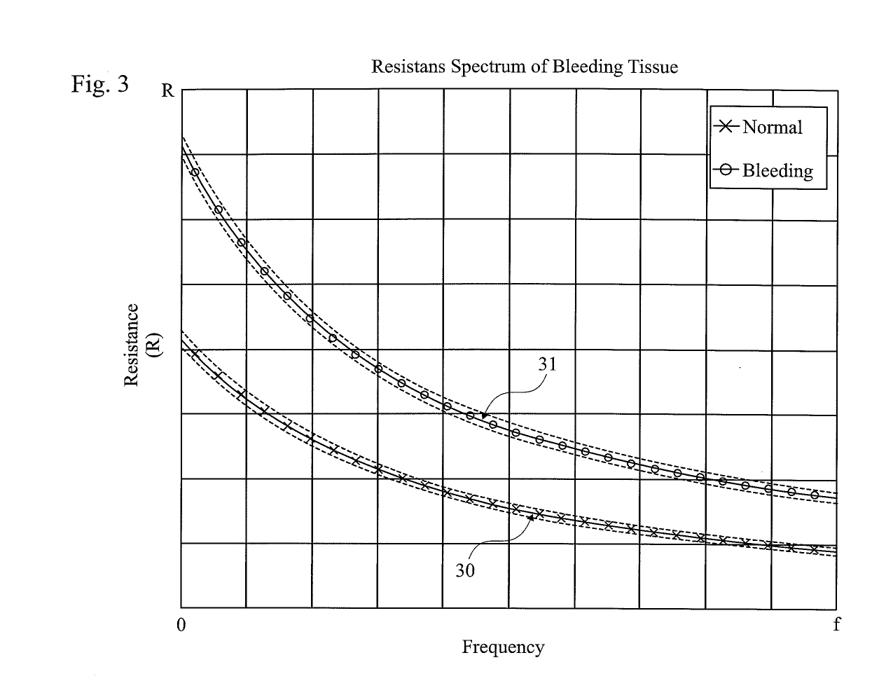

FIG. 3 illustrates a graph where the resistance (R) of normal tissue 30, and the resistance of bleeding tissue 31 are plotted against increasing current frequency into a spectrum.

In the brain the major part of the extracellular fluid consists out of cerebral spinal fluid Due to a larger resistivity of blood compared to spinal fluid leakage from blood vessels into the intracranial tissues leads to increased resistance of the measured brain part 114, as can also be seen in said shift from the lower value of normal tissue to a higher value of bleeding tissue. In fact, generally in the adult brain, more blood does not increase the size/volume of the extracellular space but increases the intracranial pressure.

Furthermore, bleeding remarkably changes the constituents of the extracellular space in the hemorrhagic region whereas the size of the cells and the composition of the intracellular fluid do not significantly change. Therefore the capacitance of the cell membranes remains relatively invariable whereas the resistivity of tissue at high frequencies changes because of the change in the extracellular fluid. At low frequencies the resistivity of tissue is determined by the resistivity of extracellular space while at high frequencies it is set by both extra- and intracellular fluids. Since the resistivity of the intracellular space does not change and the resistivity of tissue at high frequencies increases the resistance at high frequencies increases as well.

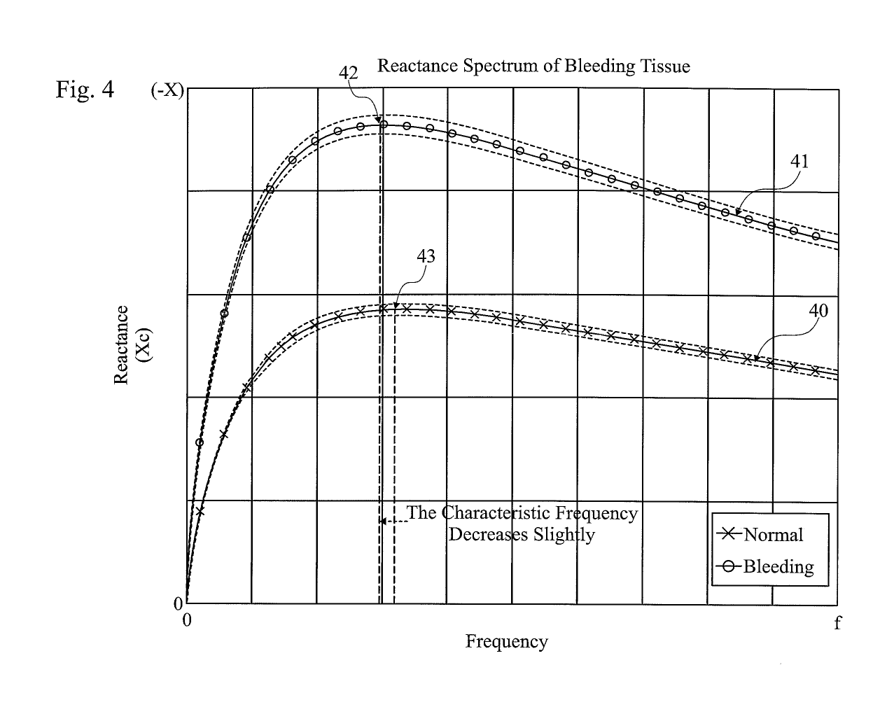

FIG. 4 illustrates a graph where the capacitive reactance (X_{C}) of normal tissue 40, and the capacitive reactance of bleeding tissue 41 are plotted against increasing current frequency into a spectrum. As seen in the graph the capacitive reactance is shifted significantly for bleeding tissue and shows higher values for all frequencies compared to normal tissue. The characteristic frequency is slightly lower for the bleeding tissue 42 compared to the normal 43.

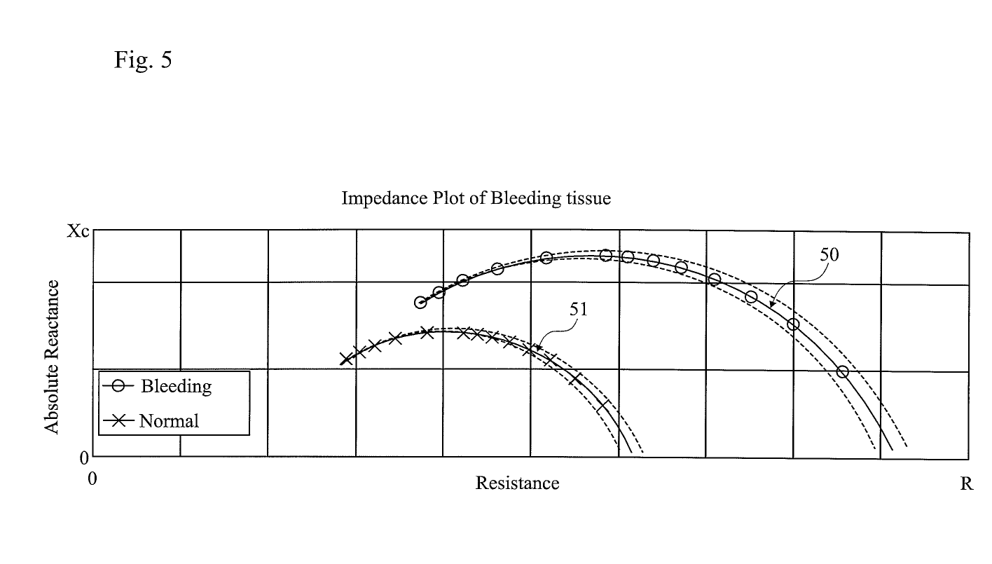

FIG. 5 illustrates an impedance plot where values for resistance (R) and values for capacitive reactance (X_{C}) of bleeding 50 and normal tissues 51 are taken from FIGS. 3 and 4 and plotted against each other into the graphs of FIG. 5. Each value plotted in the impedance plot contains the resistance and the reactance of the measured sample paired by the corresponding frequency value from FIGS. 3 and 4. i.e. the frequency of the impedance is contained in the plot plane, meaning that the graphs in FIG. 5 contain certain spectral information. The impedance points to the outmost left in said graphs correspond to the resistance/reactance at high frequency values, whereas the points to the outmost right correspond to the resistance/reactance at low frequency values.

Shown plot illustrates a notable change in the impedance profile where the changes in tissue structure as a result of bleeding cause resistance and reactance to alter as previously described. The semicircle constituting the profile of the impedance graphs is shifted to the right for bleeding tissue and its radius is increased, because the resistance increases.

In bleeding brain tissue, at low frequencies the resistance will have a major increase compared to healthy, whereas the reactance will only increase slightly. The highest increase in reactance will occur at mid-frequencies, where also the resistance changes. At high frequencies both the resistance and the reactance will increase slightly.

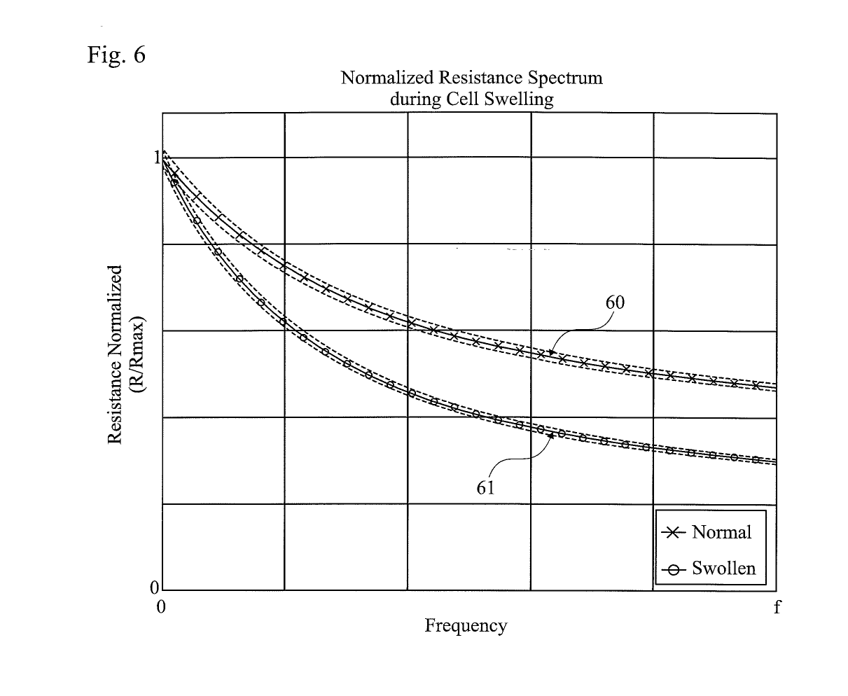

FIG. 6 illustrates a normalized resistance spectrum during cell swelling where the normalized resistance (R/Rₘₐₓ) of normal tissue 60, and the normalized resistance of tissue undergoing swelling 61 are and plotted against increasing current frequency. By normalizing the values the size factor of a brain is removed.

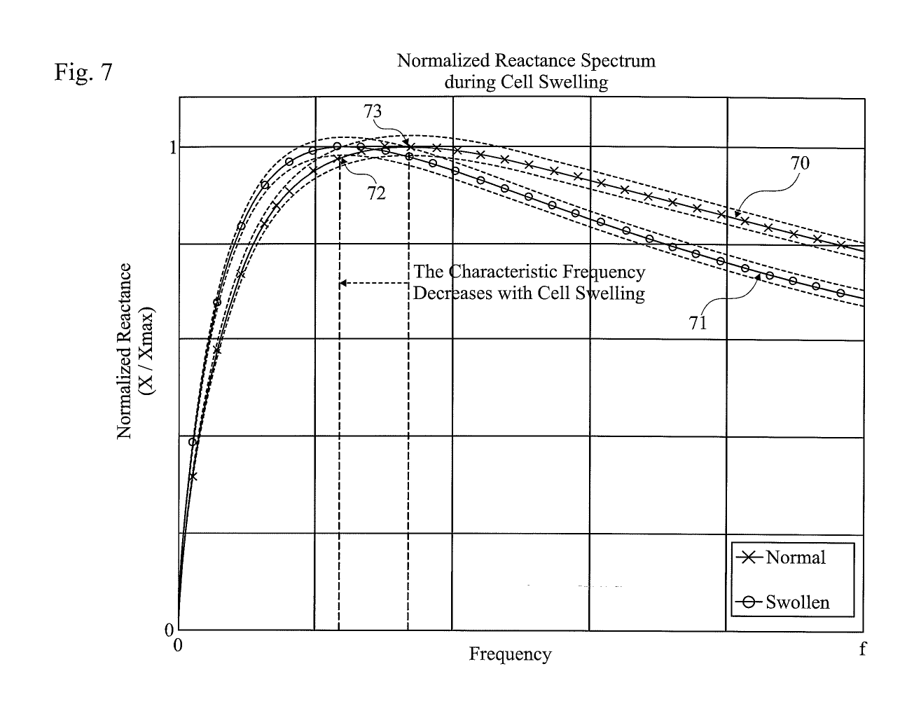

FIG. 7 illustrates a normalized reactance spectrum during cell swelling where the capacitive reactance (X_{C}/X_{Cmax}) of normal tissue 70, and the capacitive reactance of tissue undergoing swelling 71 are plotted against increasing current frequency.

The increasing of the intracellular space and the surface of the cell membrane in each cell, together with the decreasing of the extracellular space and fluid causes several changes in the dielectric properties of the tissue. The electrical conductivity of the intracellular fluid and the conductance of the intracellular space increases, the conductivity of the extracellular fluid and the conductance of the extracellular space decreases and the capacitance caused by the cell membrane of the cells increases as well. In the spectrum it is seen that the reactance increases at all frequencies, but with a certain frequency-dependency that causes a notable shift of the characteristic frequency for tissue undergoing swelling 72 towards lower frequencies compared to the characteristic frequency for normal tissue 73. This indicates that during cell swelling the electrical reactive character of the tissue increases.

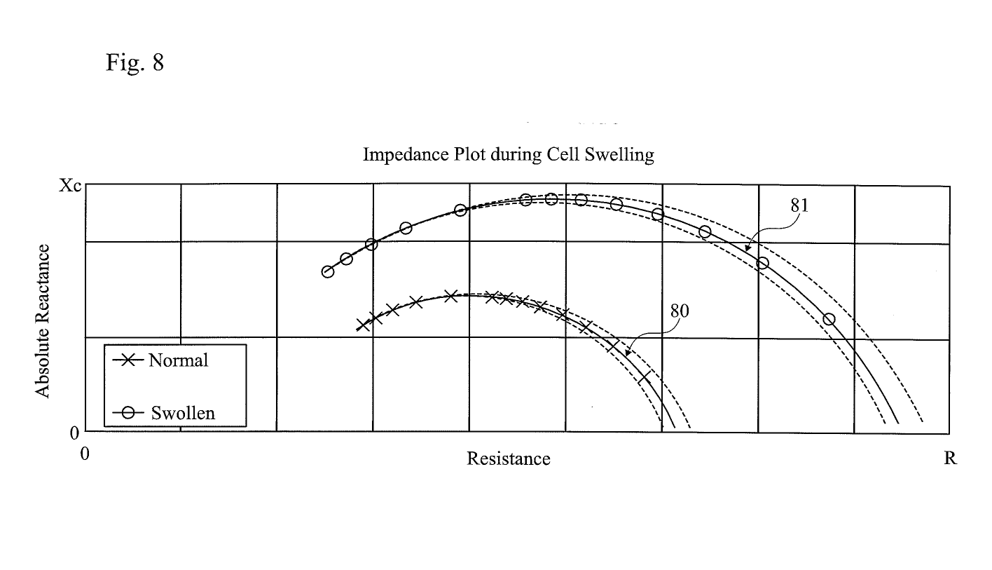

FIG. 8 illustrates an impedance plot during cell swelling where values for resistance (R) and values for capacitive reactance (X_{C}) of normal tissues 80 and of tissue undergoing cell swelling 81 are taken from FIGS. 6 and 7 and plotted against each other into the graphs of FIG. 8 in the same manner as described in FIG. 5.

As can be seen in FIG. 8 the semicircle of the impedance plot for tissue undergoing swelling is shifted to the right in the diagram, and in addition the radius is increased as a consequence of the changes in resistance and reactance described in FIGS. 6 and 7. The reactance increases for all frequencies whereas the resistance increases remarkably at low frequencies but decreases at high frequencies. During swelling the cells will take in water from the surroundings, leading to smaller extracellular space with increased resistivity, and thus a remarkable increase in resistance at low frequencies. At very high frequencies there will be a slight decrease for tissues undergoing swelling depending on that the swelling increases the intracellular space and thus decreases the resistivity of that area.

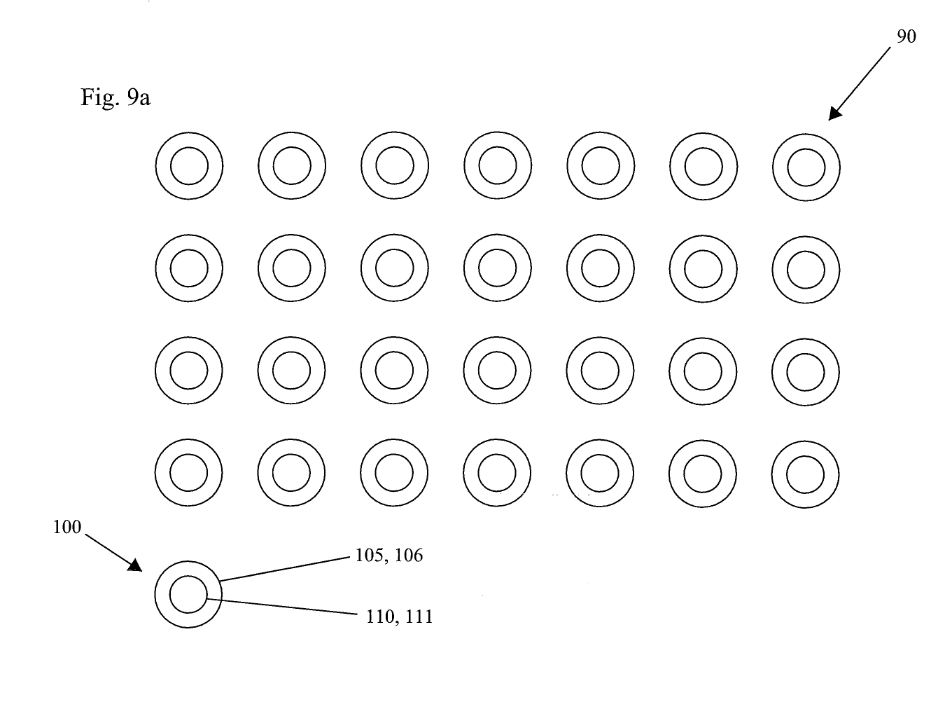

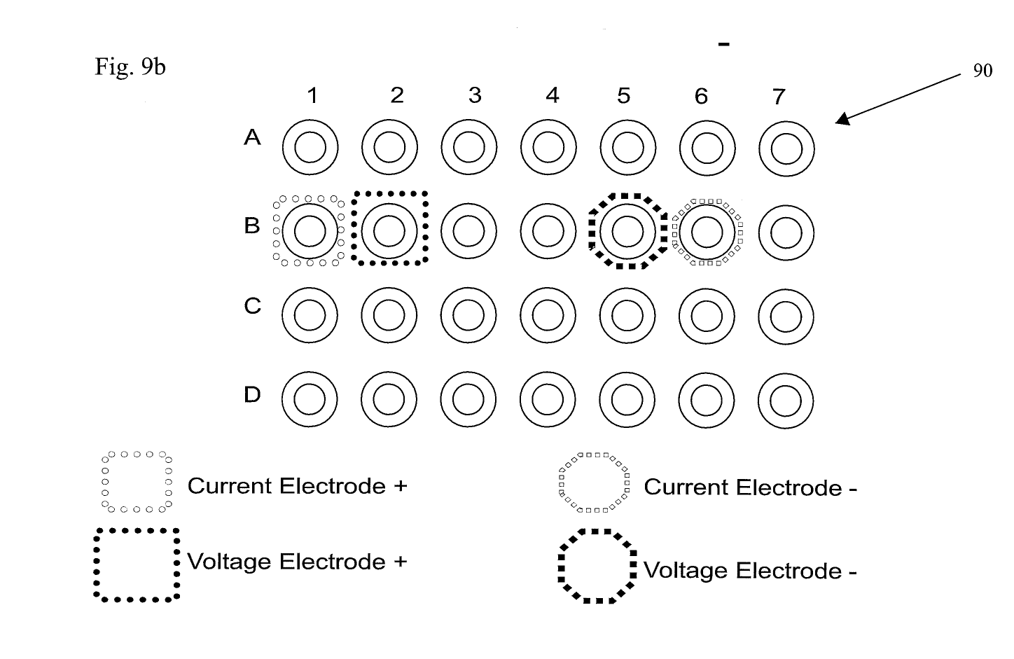

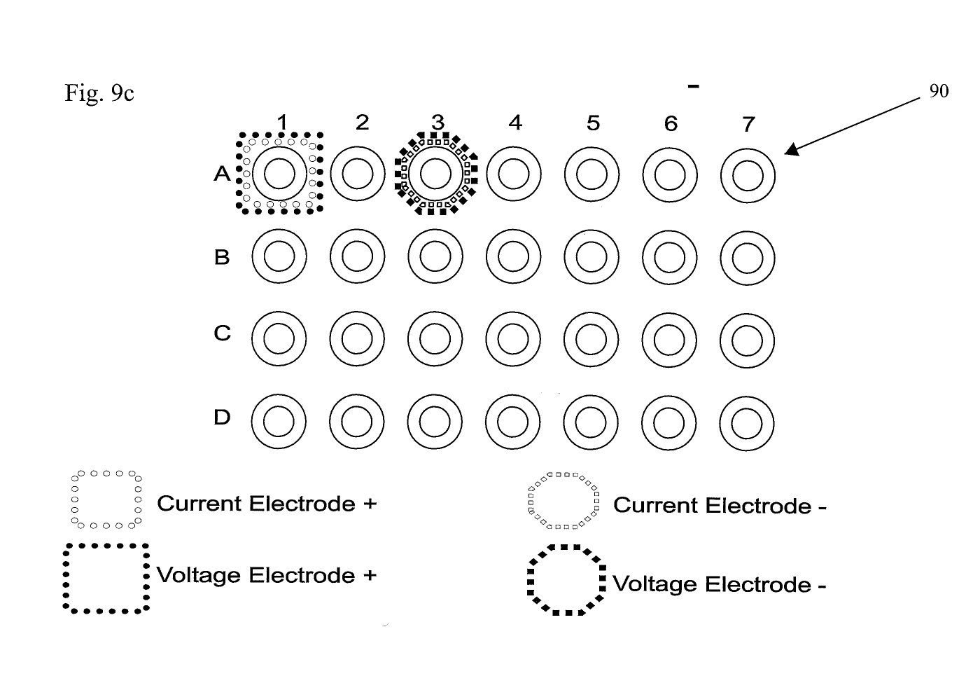

FIGS. 9a-c represent schematic views showing an example of electrode array mesh 90 which may be used for measuring bioimmitance (e.i. bioimpedance (EBI) and admittance). Said mesh 90 comprises a number of measuring units 100 evenly or unevenly distributed over the mesh, each measuring unit 100 comprising a current injecting electrode 105, 106 integrated with a potential-sensing electrode 110, 111. Evidently, it is not necessary that the electrodes are integrated. It would be possible that a measuring unit 100 comprises either of a current injecting electrode or a potential-sensing electrode.

One measurement channel is defined by the selection of two separate current injecting electrodes 105, 106 and the selection of two separate potential-sensing electrodes 110, 111, meaning any selection of two pairs of electrodes: one pair of each kind. This is illustrated in FIG. 9b where one measurement channel has been chosen. The measurement channel comprises current electrode (+) at position B1, current electrode (−) at position B6, voltage electrode (+) at position B2 and voltage electrode (−) at position B5.

Evidently more than one measurement channel may be chosen for damage detection according to the present invention, and any one of the measuring units 100 comprised by the mesh 90 may be selected. For instance, as in FIG. 9c, the measurement channel may occupy only two measuring units 100 utilizing both the current injecting electrode and the potential-sensing electrode of each unit 100.

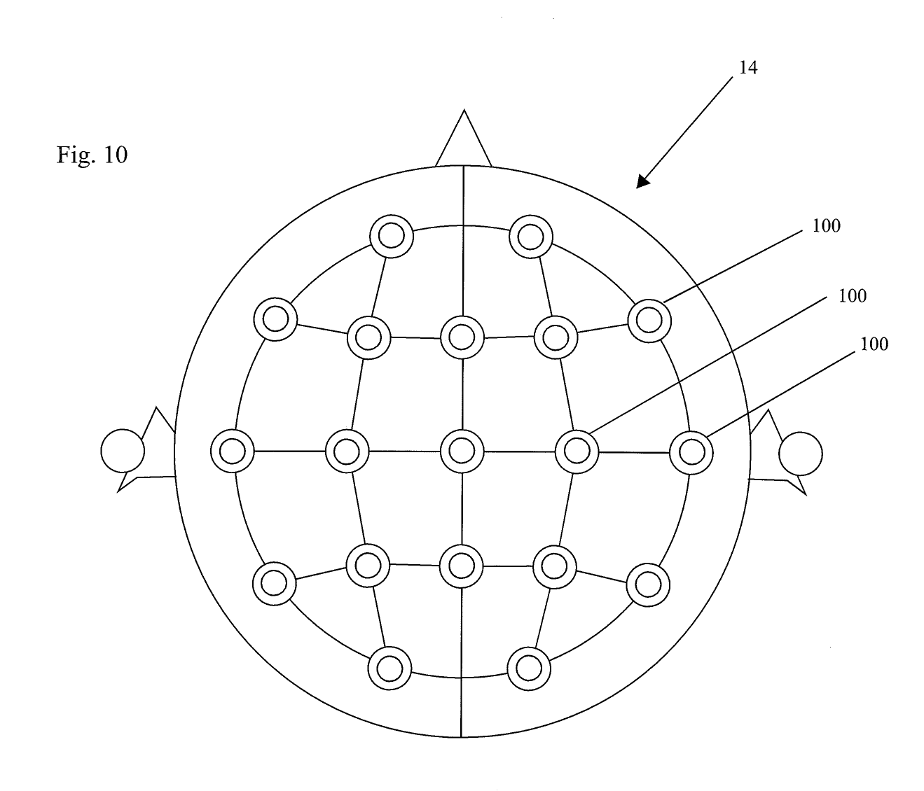

FIG. 10 is a plan view illustrating an example of a mesh of electrodes corresponding to FIGS. 9a-c in the form of a cap 14 adapted to be placed around a head of a subject 114 (human or mammal) which is to be examined. The cap 14 comprises measuring units 100, each preferably comprising two electrodes integrated into one unit: one electrode for applying electrical stimulus (e.g. current injection), and one electrode for potential sensing. Said measuring units 100 are independent and are placed in a textile garment that positions the respective electrodes upon the surface of the head. The electrodes may be for instance electrolytic, metallic or textile-based electrodes. A textile based electrode could for instance comprise metallic fibres, conductive polymers or combinations thereof.

Application of electrodes can be done directly on the surface of the skin/scalp, or with electrolytic gel between electrode and skin. As is commonly known the skin surface may also be wetted with water before applying electrodes thereto.

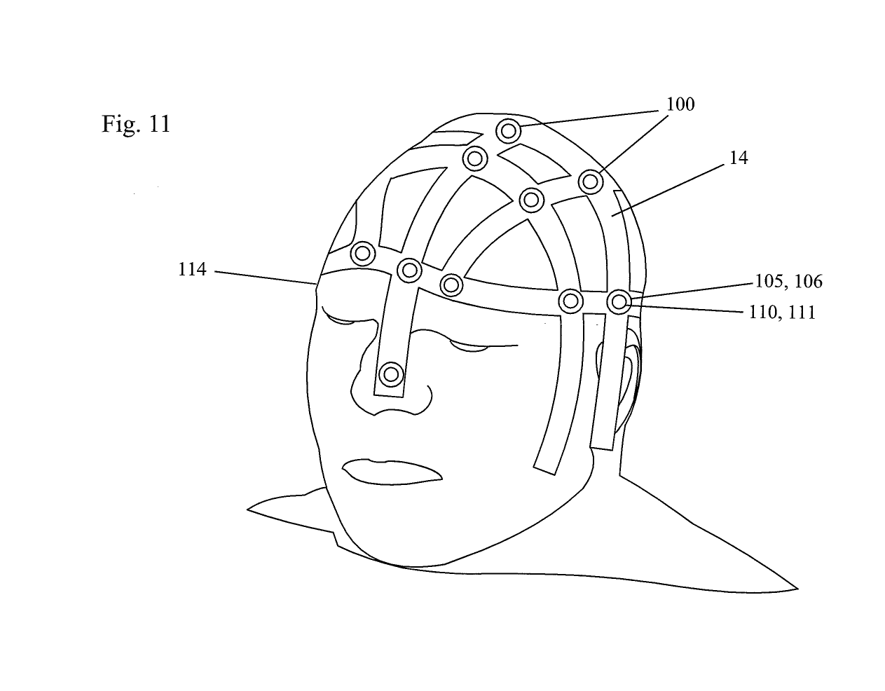

FIG. 11 shows the cap 14 in applied form placed around the periphery of the scalp 114 of a person which is to be examined by use of an apparatus according to the invention. Said mesh 90, comprising distributed measuring units 100, is positioned to surround and cover the head 114 whereupon examination is performed using measurement channels selected by the operator in charge of the procedure, or selected automatically by the system itself.

As is previously clarified in FIGS. 9a-c, each measuring unit 100 of the cap 14 preferably, but not necessarily, comprises a current electrode 105, 106 and a potential-sensing electrode 110, 111 integrated into one measuring unit.

A measurement may be performed using any number of measurement channels, depending on the current medical situation. Each measurement obtains impedance information from a volume that is defined by selected measurement channels. Therefore, by using multiple channels, it is possible to determine the location of a lesion within a brain portion that is examined. The resolution, i.e. the amount of information retrieved from a measurement, increases with increasing number of channels used for damage detection meaning the more channels that are used the better the chances of locate possible lesion. Further, a position of a lesion within a brain portion 114 may be presented to an operator either as raw-data numerically on said display 112, or in the form of an image or scan indicating the location of lesion, the latter case providing that suitable analyzing software is installed in the control unit 101 of the apparatus whereby input data may be processed and displayed in a user-friendly and easily understandable way. It is imaginable that the apparatus with which measurements are carried out comprises means for emitting light or sound signals in order to attract the attention of an operator and alert him/her of possible changes and/or abnormalities in brain tissue composition, e.g. caused by bleeding or swelling.

It is understood that the objects of the present invention set forth above, among those made apparent by the detailed description, shall be interpreted as illustrative and not in a limiting sense. Within the scope of the following claims the set-up of various alterations of the present invention may be possible, for to employ invasive electrodes instead of non-invasive ones.

Also, the portable control unit may be designed as suits a specific purpose of the use of the apparatus e.g. including intensive care, detection of perinatal asphyxia or brain stroke detection. The display of the apparatus might be embedded within the control unit or consist out of an external screen. The electrical stimulus can be injected as a current or applied as a voltage. The frequency range can be covered by sweeping or by waveforms containing multiple frequencies, e.g. multiline, white noise, step function. The impedance can be measured by one or more channels.

### BIBLIOGRAPHY

Cole, K. S. (1940). Permeability and impermeability of cell membranes for ions./Quant. Biol., 8/, 110-122.

Ollmar, S. (1998). Methods of information extraction from impedance spectra of biological tissue, in particular skin and oral mucosa—a critical review and suggestions for the future. /Bioelectrochemistry and Bioenergetics, 45/:(2), 4.

1-17. (canceled)

18. A method for detecting status of brain tissue, resulting in pathological processes affecting tissue within a portion of the brain in a mammal, including a human, the method comprising:
applying at least a first and at least a second pair of electrodes around the periphery of a head surface surrounding said portion of the brain;
generating an electrical stimulus resulting in an electrical alternating current through said brain portion;
applying said electrical stimulus between the at least first pair of electrodes;
detecting and measuring the alternating voltage developed between the at least second pair of electrodes; and
calculating the impedance of said brain portion and evaluating the result, wherein said electrical stimulus is applied between said at least first pair of electrodes at different frequencies ranging within a known spectrum,
the resistance and the reactance for each of said frequencies are measured against the corresponding frequency,
the electrical impedance of said brain portion is estimated for each one of said frequencies, and
the resistance spectrum, the reactance spectrum and/or the estimated impedance is compared to normal spectrum profiles for detection and evaluation of changes and/or abnormalities in tissue composition.

19. The method according to claim 18, wherein the frequency of said applied alternating current has a value between 1 kHz and 1 MHz.

20. The method according to claim 18, wherein the frequency of said applied alternating current has a value between 1 kHz and 2 MHz.

21. The method according to claim 18, wherein the frequency of said applied alternating current has a value between 5 kHz and 1 MHz.

22. The method according to claim 18, wherein the frequency of said applied alternating current has a value between 5 kHz and 2 MHz.

23. The method according to claim 18, wherein the frequency of said applied alternating current has a value between 20 kHz and 1 MHz.

24. The method according to claim 18, wherein the frequency of said applied alternating current has a value between 20 kHz and 2 MHz.

25. The method according to claim 18, wherein said electrical stimulus is applied through said brain portion in the form of an alternating current.

26. The method according to claim 18, wherein said electrical stimulus results in an electrical alternating current through said brain portion with an RMS value that lies within the safety limits imposed by the corresponding normative, as for example IEC60601 when applicable.

27. The method according to claim 26, wherein the amplitude of said alternating current has an RMS within the range of 0.1-1.5 mA.

28. The method according to claim 26, wherein the amplitude of said alternating current has an RMS value that is substantially invariable within at least the upper part of said frequency spectrum.

29. The method according to claim 26, wherein the amplitude of said alternating current has an RMS value that is variable within at least a lower part of said frequency spectrum.

30. An apparatus for carrying out a method for detecting status of brain tissue, resulting in pathological processes affecting tissue within a portion of the brain in a mammal, including a human, the method comprising:
applying at least a first and at least a second pair of electrodes around the periphery of a head surface surrounding said portion of the brain;
generating an electrical stimulus resulting in an electrical alternating current through said brain portion;
applying said electrical stimulus between the at least first pair of electrodes;
detecting and measuring the alternating voltage developed between the at least second pair of electrodes; and
calculating the impedance of said brain portion and evaluating the result, wherein said electrical stimulus is applied between said at least first pair of electrodes at different frequencies ranging within a known spectrum,
the resistance and the reactance for each of said frequencies are measured against the corresponding frequency,
the electrical impedance of said brain portion is estimated for each one of said frequencies, and
the resistance spectrum, the reactance spectrum and/or the estimated impedance is compared to normal spectrum profiles for detection and evaluation of changes and/or abnormalities in tissue composition, said apparatus being arranged to determine the electrical impedance and monitoring changes in electrical impedance of a brain portion, said apparatus comprising:
means capable of generating an alternating current at known current level and at known frequency to said brain portion;
at least a first pair of electrode means;
means capable of measuring the voltage drop produced across at least a second pair of electrode means as a consequence of the passage of the alternating current between said first pair of electrode means and through said brain portion; and
means for calculating the impedance in said brain portion, wherein said apparatus is arranged to calculate the electrical impedance in said brain portion as a result of changed frequency of said applied alternating current.

31. The apparatus according to claim 30, wherein said apparatus is arranged to apply an electrical stimulus through said brain portion in the form of an alternating current.

32. The apparatus according to claim 31, wherein said alternating current is applied in waveforms containing multiple frequencies, such as multisine or white noise.

33. The apparatus according to claim 30, wherein said apparatus comprises at least three pairs of electrodes distributed in a mesh arranged to be applied around the periphery of the head surface surrounding said brain portion for multichannel measurement of said brain portion.

34. The apparatus according to claim 30, wherein said apparatus comprises more than three pairs of electrodes distributed in a mesh arranged to be applied around the periphery of the head surface surrounding said brain portion for multichannel measurement of said brain portion.

35. The apparatus according to claim 33, wherein said mesh is in the form of a cap arranged to be placed around the head surface of a mammal, including a human.

36. The apparatus according to claim 35, wherein said cap is a textile cap and said electrodes used within the textile cap are electrolytic, metallic or textile-based electrodes.

37. The apparatus according to claim 33, wherein said mesh comprises at least one pair of measuring units, one measuring unit comprising one current injecting electrode integrated with one potential-sensing electrode.

38. The apparatus according to claim 33, wherein said mesh comprises more than one pair of measuring units, one measuring unit comprising one current injecting electrode integrated with one potential-sensing electrode.

39. The apparatus according to claim 30, wherein said apparatus is portable and mobile.

40. The apparatus according to claim 30, wherein said means for calculating the electrical impedance includes a central processing unit and analyzing software.

41. The apparatus according to claim 30, wherein said apparatus comprises means for graphical display.
